SEQUENCE LISTING (22 sequences)

SEQ ID NO: 1 - protein, 9 aa, Human papillomavirus
VVYRDSIPH

SEQ ID NO: 2 - protein, 9 aa, Human papillomavirus
IMCLRFLSK

SEQ ID NO: 3 - protein, 9 aa, Human papillomavirus
KCLNEILIR

SEQ ID NO: 4 - protein, 9 aa, Human papillomavirus
VDLLCHEQL

SEQ ID NO: 5 - protein, 9 aa, Human papillomavirus
CYEQLGDSS

SEQ ID NO: 6 - protein, 14 aa, Clostridium tetani
QYIKANSKFIGITE

SEQ ID NO: 7 - protein, 10 aa, Unknown
  Description of Unknown Pan DR epitope peptide
AKFVAAWTLK

SEQ ID NO: 8 - protein, 17 aa, Human adenovirus
MRYMILGLLALAAVCSA

SEQ ID NO: 9 - protein, 16 aa, Human adenovirus
RYMILGLLALAAVCSA

SEQ ID NO: 10 - protein, 17 aa, Human adenovirus
MRAAGIGILTVAAAAAG

SEQ ID NO: 11 - protein, 17 aa, Human adenovirus
MAGILGFVFTLAAAAAG

SEQ ID NO: 12 - protein, 10 aa, Human herpesvirus
SSCSSCPLSK

SEQ ID NO: 13 - protein, 9 aa, Human herpesvirus
IVTDFSVIK

SEQ ID NO: 14 - protein, 9 aa, SARS coronavirus
LYLTQDLFL

SEQ ID NO: 15 - protein, 9 aa, Human herpesvirus
GLCTLVAML

SEQ ID NO: 16 - protein, 9 aa, Hepatitis B virus
WLSLLVPFV

SEQ ID NO: 17 - protein, 13 aa, Hepatitis B virus
MQWNSTTFHQTLQ

SEQ ID NO: 18 - protein, 9 aa, Human papillomavirus
SIPHAACHK

SEQ ID NO: 19 - protein, 9 aa, Human papillomavirus
ATLQDIVLH

SEQ ID NO: 20 - protein, 8 aa, Mus musculus
SIINFEKL

SEQ ID NO: 21 - protein, 9 aa, Human papillomavirus
RAHYNIVTF

SEQ ID NO: 22 - protein, 6 aa, Artificial Sequence
  Description of Artificial Sequence Synthetic 6xHis tag
HHHHHH

(19) United States
(12) Patent Application Publication
(10) Pub. No.: US 2009/0117123 A1
(43) Pub. Date: May 7, 2009

(54) IMMUNOPEPTIDES OF HPV E6 AND E7 PROTEINS

(75) Inventors: Shih-Jen Liu, Zhunan (TW); Hsin-Wei Chen, Zhunan (TW); Pele Choi-Sing Chong, Zhunan (TW)

Correspondence Address: OCCHIUTI ROHLICEK & TSAO, LLP, 10 FAWCETT STREET, CAMBRIDGE, MA, 02138, US

(73) Assignee: National Health Research Institutes, Zhunan Town (TW)

(21) Appl. No.: 12/235,872

(22) Filed: Sep. 23, 2008

Related U.S. Application Data
(60) Provisional application No. 60/984,852, filed on Nov. 2, 2007.

(51) Int. Cl. A61K 39/395; C07K 7/06; C07K 7/08; C07K 14/00; A61P 31/12; A61K 38/08; A61K 38/10; A61K 38/16

(52) U.S. Cl. 424/139.1; 530/328; 530/327; 530/326; 530/325; 530/324; 514/12; 514/16; 514/15; 514/14; 514/13

(57) ABSTRACT

An immunopeptide containing a cytotoxic T lymphocyte epitope derived from HPV E6 or HPV E7 protein. The immunopeptide can be used as a component of a vaccine for enhancing immune responses against HPV or treating HPV-associated diseases. Also within the scope of this invention are a nucleic acid encoding the immunopeptide and an antibody specific to the CTL epitope.

### BACKGROUND OF THE INVENTION

Persistent infection with human papilloma virus (HPV) is believed to be the predominant risk factor for cervical cancer. More specifically, HPV E6 and HPV E7 proteins are found to be oncogenic.

Anti-HPV vaccines that contain immunopeptides derived from HPV E6 or HPV E7 are being developed for controlling cervical cancer. They enhance host immune responses against either oncoproteins, thereby blocking oncogenesis. To develop such vaccines, identifying immonopeptides is the most challenging task.

### SUMMARY OF THE INVENTION

This invention is based on the unexpected discovery that five novel peptides, three derived from HPV E6 and two from HPV E7, induce strong cytotoxic T lymphocyte (CTL) responses in a human HLA A11 transgenic mouse.

In one aspect, the present invention features an isolated immunopeptide having 8-50 amino acid residues. The immunopeptide includes an amino acid sequence of VVYRDSIPH (SEQ ID NO:1), IMCLRFLSK (SEQ ID NO:2), KCLNEILIR (SEQ ID NO:3), VDLLCHEQL (SEQ ID NO:4), or CYEQLGDSS (SEQ ID NO:5). It can further include another T-cell epitope, such as QYIKANSKFIGITE (tetanus toxoid 830-843; SEQ ID NO:6) and AKFVAAWTLK (Pan DR epitope; SEQ ID NO:7). Alternatively or in addition, the immunopeptide further includes an endoplasmic reticulum target sequence, e.g.,

| | |
|---|---|
| MRYMILGLLALAAVCSA, | (SEQ ID NO:8) |
| RYMILGLLALAAVCSA, | (SEQ ID NO:9) |
| MRAAGIGILTVAAAAAG, | (SEQ ID NO:10) |
| and | |
| MAGILGFVFTLAAAAAG. | (SEQ ID NO:11) |

The term “isolated” used in the phase “an isolated immunopeptide” refers to an immunopeptide substantially free from naturally associated molecules; namely, it is at least 75% (i.e., any number between 75% and 100%, inclusive) pure by dry weight. Purity can be measured by any appropriate method, e.g., column chromatography, polyacrylamide gel electrophoresis, and HPLC. An immunopeptide is capable of inducing immune responses, such as CTL responses or antibody formation, when introduced to a subject.

In another aspect, this invention features an isolated nucleic acid that includes a nucleotide sequence encoding any of the immunopeptides described above. It further features a cell containing the nucleic acid, which can be a vector allowing expression of any of the immunopeptides. The term “isolated” used in the phase “an isolated nucleic acid” refers to a nucleic acid substantially free from naturally associated molecules; namely, it is at least 75% (i.e., any number between 75% and 100%, inclusive) pure by dry weight.

Further, the present invention provides a method of treating a HPV-associated disease, e.g., HPV infection and cervical cancer, by administering to a subject in need of the treatment an effective amount of a composition containing either one of the above-described immunopeptides, or a DNA plasmid expressing it. The composition, preferably also containing a carrier (e.g., adjuvant), enhances anti-HPV immune responses in the subject.

The term “treating” as used herein refers to the application or administration of a composition including one or more active agents to a subject, who has a HPV-associated disease, a symptom of the disease, or a predisposition toward the disease, with the purpose to cure, heal, alleviate, relieve, alter, remedy, ameliorate, improve, or affect the disease, the symptoms of the disease, or the predisposition toward the disease. “An effective amount” as used herein refers to the amount of each active agent required to confer therapeutic effect on the subject, either alone or in combination with one or more other active agents. Effective amounts vary, as recognized by those skilled in the art, depending on route of administration, excipient usage, and co-usage with other active agents.

Also with the scope of this invention is an antibody that specifically binds to one of the five following peptide: VVYRDSIPH (SEQ ID NO:1), IMCLRFLSK (SEQ ID NO:2), KCLNEILIR (SEQ ID NO:3), VDLLCHEQL (SEQ ID NO:4), and CYEQLGDSS (SEQ ID NO:5).

In addition, this invention features the use of any of the immunopeptides or a DNA plasmid expressing the immunopeptide for the manufacture of a medicament for the treatment of HPV infection or a disease associated with HPV infection.

Other features or advantages of the present invention will be apparent from the following drawings and detailed description of several embodiments, and also from the appending claims.

### BRIEF DESCRIPTION OF THE DRAWINGS

FIG. 1 is a photograph showing Coomassie blue-stained protein bands on a 15% SDS-polyacrylamide gel. Left panel: SDS-PAGE analysis of purified and unpurified HLA A11 heavy chain expressed in E. coli. Lane 1: lysate of E. coli cells not induced by isopropyl b-D-thiogalactopyranoside (IPTG); Lane 2: lysate of E. coli cells induced by IPTG induction; Lane 3: supernatant obtained from centrifuging lysate of IPTG-induced E. coli cells; Lanes 4 and 5: fractions obtained from wishing a Ni-NTA column with 2 M urea; Lanes 6 and 7: HLA A-11 heavy chain purified by chromatography with a Ni-NTA-agarose column. Right panel: SDS-PAGE analysis of purified and unpurified β2-microglobulin expressed in E. coli. Lane 8: lysate of E. coli cells not induced by IPTG; Lane 9: lysate of E. coli cells induced by IPTG; Lane 10: supernatant obtained from centrifuging lysate of IPTG-induced E. coli cells; Lanes 11 and 12: fractions obtained from washing a Ni-NTA column with 2 M urea; Lanes 13: β2-microglobulin purified by chromatography using a Ni-NTA-agarose column.

FIG. 2 is an illustration of MHC-peptide complex formation assay.

FIG. 3 is a diagram showing the binding activity of HLA A2-restricted peptides (panel A) and HLA A11-restricted peptides (panel B) to their corresponding MHC molecules.

FIG. 4 is a diagram showing the relative binding activity of 9-mer peptides derived from E6 and E7 proteins of HPV16, HPV18, HPV58, and HPV52 to HLA A11-β2 microglobulin. Relative binding (%)=(sample O.D.−negative control O.D.)/(positive control O.D.−negative control O.D.)%

FIG. 5A is a diagram showing interferon-γ (IFN-γ) secretion of splenocytes obtained from HLA-A11 transgenic mice immunized with peptide VVYRDSIPH (VVY; SEQ ID NO:1) or a control peptide.

FIG. 5B is a diagram showing IFN-γ secretion of splenocytes obtained from HLA-A24 transgenic mice immunized with peptide CYEQLGDSS (A24 CYE, SEQ ID NO:5) or a control peptide.

FIG. 6A is a diagram showing peptide-tetramer staining of CD8⁺ T cells in HLA-A11 transgenic mice immunized with peptide VVY or a control peptide.

FIG. 6B is a chart showing cytotoxic T lymphocyte effect induced by peptide VVY in splenocytes obtained from VVY immunized HLA-A11 transgenic mice.

FIG. 7A is a diagram showing IFN-γ secretion of splenocytes obtained from HLA-A11 transgenic mice immunized with a DNA plasmid expressing a fusion protein containing the H-2K^{b} restricted CTL epitope SIINFEKL (SII; SEQ ID NO:20), and the HPV18E6E7 polypeptide, or control plasmid pCIneo. Splenocytes derived from the immunized transgenic mice were stimulated with peptides VVYRDSIPH (VVY; SEQ ID NO:1), SIPHAACHK (SIP; SEQ ID NO:18), ATLQDIVLH (ATL; SEQ ID NO:19), SII, and a control peptide RAHYNIVTF (SEQ ID NO:21). The levels of IFN-γ secretion of the splenocytes were determined via the Enzyme-linked immunospot (ELISPOT) assay.

FIG. 7B is a chart showing cytotoxic T lymphocyte effects induced by the DNA plasmids described above.

### DETAILED DESCRIPTION OF THE INVENTION

This invention features an immunopeptide having about 8-50 amino acid residues. The immunopeptide includes a CTL epitope derived from HPV E6 or E7 protein and restricted to a particular HLA class I allele. A CTL epitope refers to a peptide capable of activating a CTL (also known as Tc or killer T cell), which subsequently stimulates CTL responses, i.e., inducing death of abnormal cells (e.g., virus-infected or tumor cells). A CTL epitope, typically including 8-11 amino acid residues, forms a complex with a particular MHC class I molecule (including a heavy chain and a β2 microglobulin) presented on the surface of an antigen-presenting cell. This complex, upon binding to a T cell receptor of a CD₈ T cell (a CTL), activates the T cell, thus triggering CTL responses.

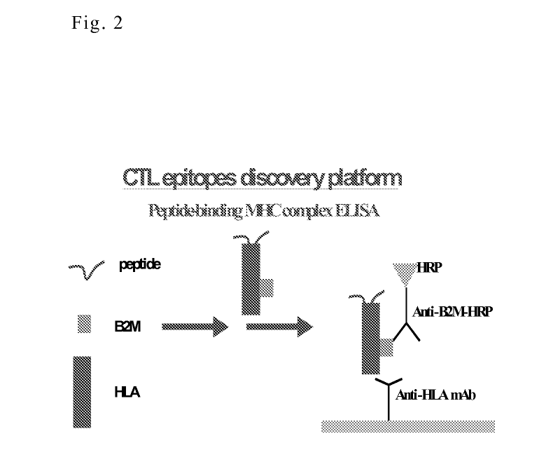

CTL epitopes derived from HPV E6 and E7 proteins can be identified as follows. The amino acid sequences of E6 and E7 are searched by a computational program well known in the art to look for potential CTL epitopes contained therein. When such epitopes are identified, peptides (e.g., 8-12 aa) containing them are synthesized and subjected to determination of their HLA class I restriction by assays known in the art. In one example, an MHC-peptide complex formation assay is performed as follows to determine the HLA class I restriction of a peptide. See FIG. 2. A heavy chains encoding by an HLA class I allele (e.g., HLA-A11 or HLA-A24) and β2-microglobulin are expressed and purified. They are then mixed with one of the synthetic peptides mentioned above. Any MHC-peptide complex thus formed can be detected by, e.g., ELISA. It is well known that a heavy chain encoded by a particular HLA class I allele forms stable complex with β2 microglobulin only in the presence of a peptide restricted to the HLA class I allele. Therefore, the formation of the MHC-peptide complex, detected by, e.g., ELISA, indicates that the synthetic peptide contains an epitope restricted to the HLA class I allele.

After a synthetic peptide is determined to be restricted to a particular HLA class I allele, it can then be subjected to in vitro or in vivo assays to confirm whether it contains a CTL epitope. An example of an in vitro assay follows. A human carrying the HLA class I allele is identified by genotyping using methods known in the art. His or her peripheral blood mononuclear cells (PBMC) are collected and exposed to the peptide in the presence of autologous antigen presenting cells. If the synthetic peptide activates the PBMCs, it indicates that the epitope included therein is a CTL epitope restricted to the HLA class I allele. In another example, an in vivo assay described below is employed to determine whether the synthetic peptide includes a CTL epitope. A transgenic mouse expressing the HLA class I allele is immunized with the synthetic peptide. Induction of CTL immune responses (e.g., secretion of cytokines such as IFN-γ and IL-2) indicates that the peptide contains a CTL peitope restricted to the HLA class I allele.

The immunopeptide of this invention can further include another T cell epitope, e.g., QYIKANSKFIGITE (tetanus toxoid 830-843; SEQ ID NO:6) or AKFVAAWTLK (a pan DR epitope; SEQ ID NO:7). Alternatively or in addition, it can further include an endoplasmic reticulum target sequence. This target sequence facilitates entrance of a polypeptide containing it into the class I antigen presentation pathway, in which CTL epitopes of the polypeptide form complexes with HLA class I molecules. Examples of the target sequence include but are not limited to the peptides MRYMILGLLALAAVCSA (SEQ ID NO:8) and RYMILGLLALAAVCSA (SEQ ID NO:9), both derived from adenovirus E3 protein), peptide MRAAGIGILTVAAAAAG (SEQ ID NO:10; see Minev et al. 2000, Eur J Immunol. 30(8):2115-24.) and peptide MAGILGFVFTLAAAAAG (SEQ ID NO:11; see Gueguen, et al., 1994, J Exp Med. 1994, 180(5):1989-94).

The immunopeptide of the invention can be obtained as a synthetic polypeptide or a recombinant polypeptide. To prepare a recombinant polypeptide, a nucleic acid encoding it can be linked to another nucleic acid encoding a fusion partner, e.g., Glutathione-S-Transferase (GST), 6x-His tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant immunopeptide of this invention.

As this immunopeptide contains a CTL epitope derived from HPV E6 or E7 protein, it can be used to prepare an immunogenic composition (e.g., a vaccine) for enhancing CTL immune responses against HPV. Upon administration to a subject, preferably carrying HLA A11, HLA A24, or an equivalent HLA allele, this composition is effective in treating diseases induced by HPV, e.g., HPV infection and cervical cancer. An equivalent HLA allele of HLA A11 or HLA A24 is an allele that cross-reacts with a peptide restricted to HLA A11 or HLA A24, e.g., HLA-A3 and HLA-A2.

To prepare the just-mentioned immunogenic composition, the immunopeptides may first require chemical modification since they may not have a sufficiently long half-life. A chemically modified peptide or a peptide analog includes any functional chemical equivalent of the peptide characterized by its increased stability and/or efficacy in vivo or in vitro in respect of the practice of the invention. The term peptide analog also refers to any amino acid derivative of a peptide as described herein. A peptide analog can be produced by procedures that include, but are not limited to, modifications to side chains, incorporation of unnatural amino acids and/or their derivatives during peptide synthesis and the use of cross-linkers and other methods that impose conformational constraint on the peptides or their analogs. Examples of side chain modifications include modification of amino groups, such as by reductive alkylation by reaction with an aldehyde followed by reduction with NaBH₄; amidation with methylacetimidate; acetylation with acetic anhydride; carbamylation of amino groups with cyanate; trinitrobenzylation of amino groups with 2, 4, 6, trinitrobenzene sulfonic acid (TNBS); alkylation of amino groups with succinic anhydride and tetrahydrophthalic anhydride; and pyridoxylation of lysine with pyridoxa-5′-phosphate followed by reduction with NABH₄. The guanidino group of arginine residues may be modified by the formation of heterocyclic condensation products with reagents such as 2,3-butanedione, phenylglyoxal and glyoxal. The carboxyl group may be modified by carbodiimide activation via o-acylisourea formation followed by subsequent derivatization, for example, to a corresponding amide. Sulfhydryl groups may be modified by methods, such as carboxymethylation with iodoacetic acid or iodoacetamide; performic acid oxidation to cysteic acid; formation of mixed disulphides with other thiol compounds; reaction with maleimide; maleic anhydride or other substituted maleimide; formation of mercurial derivatives using 4-chloromercuribenzoate, 4-chloromercuriphenylsulfonic acid, phenylmercury chloride, 2-chloromercuric-4-nitrophenol and other mercurials; carbamylation with cyanate at alkaline pH. Tryptophan residues may be modified by, for example, oxidation with N-bromosuccinimide or alkylation of the indole ring with 2-hydroxy-5-nitrobenzyl bromide or sulphonyl halides. Tryosine residues may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

The composition can be prepared, e.g., according to the method described in any methods known in the art. The composition contains an effective amount of the immunopeptide of the invention, and a pharmaceutically acceptable carrier such as a phosphate buffered saline, a bicarbonate solution, or an adjuvant. The carrier is selected on the basis of the mode and route of administration, and standard pharmaceutical practice. Suitable pharmaceutical carriers and diluents, as well as pharmaceutical necessities for their use, are described in Remington's Pharmaceutical Sciences. An adjuvant, e.g., a cholera toxin, Escherichia coli heat-labile enterotoxin (LT), liposome, immune-stimulating complex (ISCOM), or immunostimulatory sequences oligodeoxynucleotides (ISS-ODN), can also be included in a composition of the invention, if necessary. The composition can also include a polymer that facilitates in vivo delivery. See Audran R. et al. Vaccine 21:1250-5, 2003; and Denis-Mize et al. Cell Immunol., 225:12-20, 2003. In one example, the immunopeptide is a component of a multivalent composition of vaccine against HPV. This multivalent composition contains at least one immunopeptide described above, along with at least one protective antigen isolated from influenza virus, para-influenza virus 3, Strentococcus pneumoniae, Branhamella (Moroxella) gatarhalis, Staphylococcus aureus, or respiratory syncytial virus, in the presence or absence of adjuvant. In another example, the immunopeptide is formulated as a virosome, which contains functional viral envelope glycoproteins, such as influenza virus hemagglutinin (HA).

Methods for preparing vaccines are generally well known in the art, as exemplified by U.S. Pat. Nos. 4,601,903; 4,599,231; 4,599,230; and 4,596,792. Vaccines may be prepared as injectables, as liquid solutions or emulsions. The immuopeptide of this invention may be mixed with physiologically acceptable and excipients compatible. Excipients may include, water, saline, dextrose, glycerol, ethanol, and combinations thereof. The vaccine may further contain minor amounts of auxiliary substances such as wetting or emulsifying agents, pH buffering agents, or an adjuvant to enhance the effectiveness of the vaccines. Methods of achieving adjuvant effect for the vaccine includes use of agents, such as aluminum hydroxide or phosphate (alum), commonly used as 0.05 to 0.1 percent solutions in phosphate buffered saline. Vaccines may be administered parenterally, by injection subcutaneously or intramuscularly. Alternatively, other modes of administration including suppositories and oral formulations may be desirable. For suppositories, binders and carriers may include, for example, polyalkalene glycols or triglycerides. Oral formulations may include normally employed incipients such as, for example, pharmaceutical grades of saccharine, cellulose, magnesium carbonate and the like. These compositions take the form of solutions, suspensions, tablets, pills, capsules, sustained release formulations or powders and contain 10-95% of the immunopeptide described herein.

The vaccine of this invention can also be a dendritic cell-based vaccine, which contains dendritic cells pulsed with any of the immunopeptides described herein. Methods for preparing dendritic cell-based vaccines are well known in the art. See Slingluff et al., Clin Cancer Res. 12:2342s-2345s, 2006; Buchsel et al. Clin J Oncol Nurs. 10:629-40, 2006; and Yamanaka et al., Expert Opin Biol Ther.7:645-9, 2007.

The vaccine is administered in a manner compatible with the dosage formulation, and in an amount that is therapeutically effective, protective and immunogenic. The quantity to be administered depends on the subject to be treated, including, for example, the capacity of the individual's immune system to synthesize antibodies, and if needed, to produce a cell-mediated immune response. Precise amounts of active ingredient required to be administered depend on the judgment of the practitioner. However, suitable dosage ranges are readily determinable by one skilled in the art and may be of the order of micrograms of the polypeptide of this invention. Suitable regimes for initial administration and booster doses are also variable, but may include an initial administration followed by subsequent administrations. The dosage of the vaccine may also depend on the route of administration and varies according to the size of the host.

A subject susceptible to HPV infection can be identified and administered the immunogenic composition described above. The dose of the composition depends, for example, on the particular immunopeptide, whether an adjuvant is co-administered with the immunopeptide, the type of adjuvant co-administered, the mode and frequency of administration, as can be determined by one skilled in the art. Administration is repeated, if necessary, as can be determined by one skilled in the art. For example, a priming dose can be followed by three booster doses at weekly intervals. A booster shot can be given at 4 to 8 weeks after the first immunization, and a second booster can be given at 8 to 12 weeks, using the same formulation. Sera or T-cells can be taken from the subject for testing the immune response elicited by the vaccine against the HPV E6 or E7 protein. Methods of assaying cytotoxic T cells against a protein or infection are well known in the art. Additional boosters can be given as needed. By varying the amount of the immunopeptide, the dose of the composition, and frequency of administration, the immunization protocol can be optimized for eliciting a maximal immune response. Before a large scale administering, efficacy testing is desirable. In an efficacy testing, a non-human subject can be administered via an oral or parenteral route with a composition of the invention. After the initial administration or after optional booster administration, both the test subject and the control subject (receiving mock administration) are challenged with an HPV. End points other than lethality can be used. Efficacy is determined if subjects receiving the composition are free from HPV infection or develop symptoms associated with HPV infection at a rate lower than control subjects. The difference should be statistically significant.

The immunopeptide of this invention can also be used to generate antibodies in animals (for production of antibodies) or humans (for treatment of diseases). Methods of making monoclonal and polyclonal antibodies and fragments thereof in animals are known in the art. See, for example, Harlow and Lane, (1988) Antibodies: A Laboratory Manual, Cold Spring Harbor Laboratory, New York. The term “antibody” includes intact molecules as well as fragments thereof, such as Fab, F(ab′)₂, Fv, scFv (single chain antibody), and dAb (domain antibody; Ward, et. al. (1989) Nature, 341, 544). These antibodies can be used for detecting the HPV E6 or E7 protein, e.g., in determining whether a test sample from a subject contains HPV.

In general, to produce antibodies against a peptide, the peptide can be coupled to a carrier protein, such as KLH, mixed with an adjuvant, and injected into a host animal. Antibodies produced in the animal can then be purified by peptide affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

Polyclonal antibodies, heterogeneous populations of antibody molecules, are present in the sera of the immunized subjects. Monoclonal antibodies, homogeneous populations of antibodies to a polypeptide of this invention, can be prepared using standard hybridoma technology (see, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sci. USA 80, 2026, and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production. In addition, techniques developed for the production of “chimeric antibodies” can be used. See, e.g., Morrison et al. (1984) Proc. Natl. Acad. Sci. USA 81, 6851; Neuberger et al. (1984) Nature 312, 604; and Takeda et al. (1984) Nature 314:452. A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage library of single chain Fv antibodies. Single chain antibodies are formed by linking the heavy and light chain fragments of the Fv region via an amino acid bridge. Moreover, antibody fragments can be generated by known techniques. For example, such fragments include, but are not limited to, F(ab′)₂ fragments that can be produced by pepsin digestion of an antibody molecule, and Fab fragments that can be generated by reducing the disulfide bridges of F(ab′)₂ fragments. Antibodies can also be humanized by methods known in the art. For example, monoclonal antibodies with a desired binding specificity can be commercially humanized (Scotgene, Scotland; and Oxford Molecular, Palo Alto, Calif.). Fully human antibodies, such as those expressed in transgenic animals are also features of the invention (see, e.g., Green et al. (1994) Nature Genetics 7, 13; and U.S. Pat. Nos. 5,545,806 and 5,569,825).

This invention also features an isolated nucleic acid encoding the immunopeptide of this invention, including a vector allowing expression of the immunopeptide. Such a nucleic acid can be used, as a DNA vaccine, for immunization by administration of the nucleic acid directly to a subject via a live vector, such as Salmonella, BCG, adenovirus, poxvirus, vaccinia, or a non-viral vector. Immunization methods based on nucleic acids are well known in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### EXAMPLE 1

### Identification of HLA A11 Restricted CTL Epitopes Derived from HPV E6 Proteins

Five computational programs, available from the websites listed below, were applied to identify potential CTL-epitopes from E6 proteins of HPV 16 and 18 (predominant in western countries), and HPV 52, and 58 (Asian-specific serotypes).
bimas.cit.nih.gov/molbio/hla_bind/;
syfpeithi.de/Scripts/MHCServer.dll/EpitopePredction.htm;
-bs.informatik.uni-tuebingen.de/Services/SVMHC;
bio.deci.harvard.edu/Tools/rankpep.html;
tools.immuneepitope.org/analyze/html/mhc_binding.html.

The CTL epitopes thus identified were synthesized and subjected to the MHC-peptide complex formation assay as described below to determine whether they were restricted to HLA A-11.

The human HLA A11 heavy chain was expressed in E. coli as a His-Tag fusion protein following the procedures described below. An expression vector encoding the fusion protein was introduced into E. coli BL2 (DE3). A transformant thus produced was incubated at 37° C. 0.1 mM isopropyl b-D-thiogalactopyranoside (IPTG) was added to the E. coli culture when its O.D. value reached 0.5. After further culturing at 37° C. for 3-4 hours, the E. coli cells were harvested by centrifugation at 6000 rpm for 20 minutes. The cell pellets were suspended in Buffer A containing 8 M urea, 20 mM HEPES (pH 8.0), and 50 mM NaCl, and then subjected to sonication. The sonicated cells were again centrifuged and the supernant thus formed was collected and loaded on a Ni-NTA-agarose column to purify the His-Tag fused HLA A-11 heavy chain.

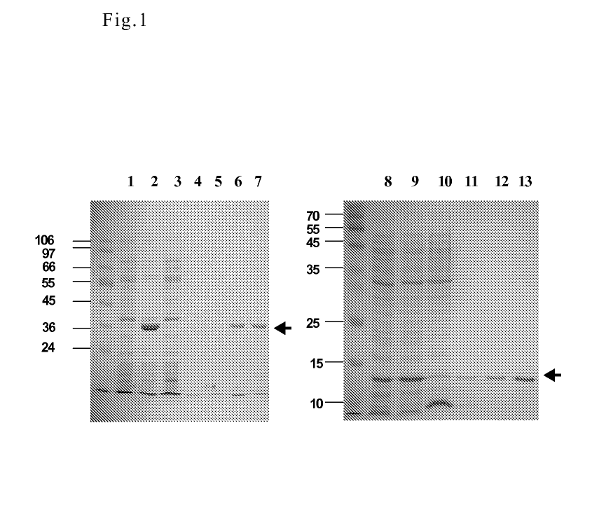

The human HLA A2 heavy chain, and His-Tag fused human β2-microglobulin were expressed and purified following the same procedures described above. As shown in FIG. 1, either the purified HLA A11 heavy chain or the purified β2-microglobulin yields a single Coomassie blue-stained band in 15% SDS-PAGE.

The accuracy of the MHC-peptide complex formation assay was first determined. The purified HLA A11 heavy chain (3 μM), β2-microglobulin (1 μM) and two positive control peptides and one negative control peptide were dissolved in a refolding buffer containing 100 mM Tris-Hcl (pH8), 400 mM L-arginine, 2 mM EDTA, 5 mM reduced glutathione, 0.5 mM oxidized glutathione and then incubated at 4° C. for 72 hours. The two positive control peptides were SSCSSCPLSK (SSC; SEQ ID NO:12) derived from EBV LMP2 protein and IVTDFSVIK (IVT; SEQ ID NO:13) derived from EBV EBNA-3B protein. The negative control peptide was LYLTQDLFL (SEQ ID NO:14), derived from SARS CoV spike protein. The HLA A11-β2-peptide complexes formed during incubation were concentrated by Centricon (Millipore Co.) and then centrifuged to remove aggregates also formed in this process. Following the same procedures described above, HLA A2 and β2-microglobulin were mixed with two A2-restricted peptides, A2GLC (GLCTLVAML, SEQ ID NO:15) and A2WLS (WLSLLVPFV, SEQ ID NO:16) or a control peptide to form A2-β2-peptide complexes.

The MHC-peptide complexes described above were then detected by the following method. An ELISA plate coated with anti-HLA antibody W6/32 (HB-95; ATCC.) (50 μL at 5 μg/ml in 100 mM carbonate buffer, pH 9.6 at 4° C. overnight). The plate was blocked with 250 ml/well 5% w/v skim milk powder in PBS at room temperature for 2 hours, and then washed twice with 300 ul/well 0.05% Tween-20 (Sigma) in PBS. The complexes were diluted in a PBS solution containing 1% BSA and subsequently added to the antibody-coated plate. The plate was incubated for 2 hours at room temperature to allow the complexes to bind to the anti-HLA antibody. After incubation, the plate was washed twice and then added with 100 μl/well horseradish peroxidase (HRP) labeled rabbit antihuman β2-microtubolin antibody (DAKO, Japan; diluted at 1:2500 in a PBS solution containing 1% BSA). The plate was incubated at room temperature for 2 hours, washed six times with PBS/0.05% Tween 20, and then added with HRP-conjugated anti-rabbit antibodies (1:2000). After incubating at room temperature for one hour, the plate was added with 3,3′-5,5′-tetramethylbenzidine hydrogenperoxide (TMB, Sigma), incubated for 30 minutes, and levels of the color thus developed in each well were read at 450 nM using an ELISA reader.

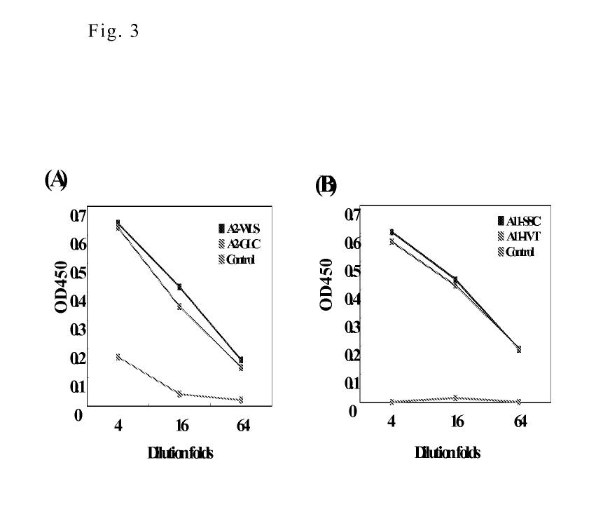

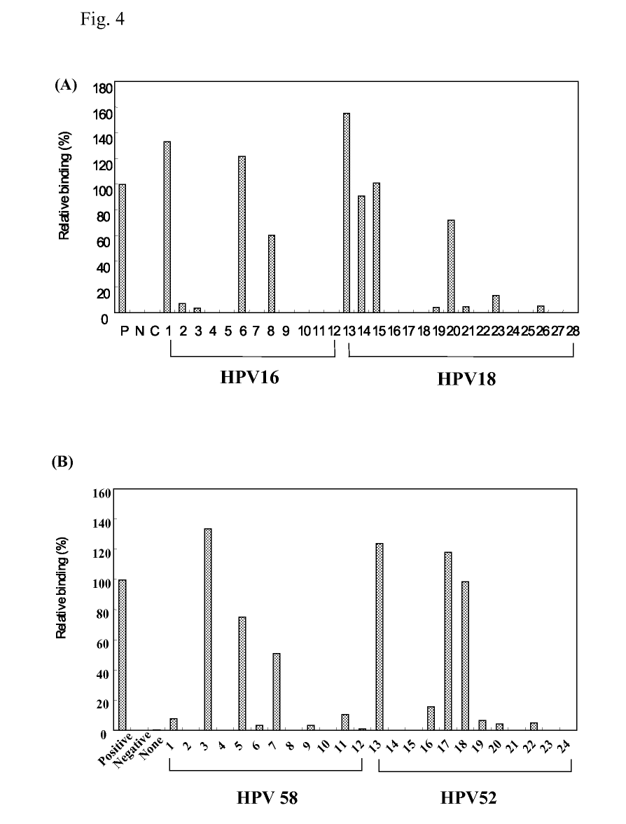

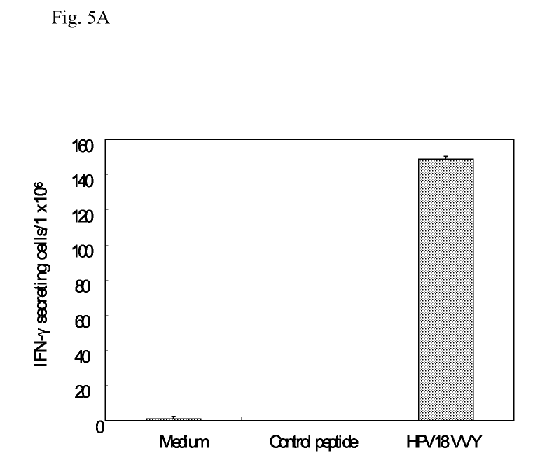

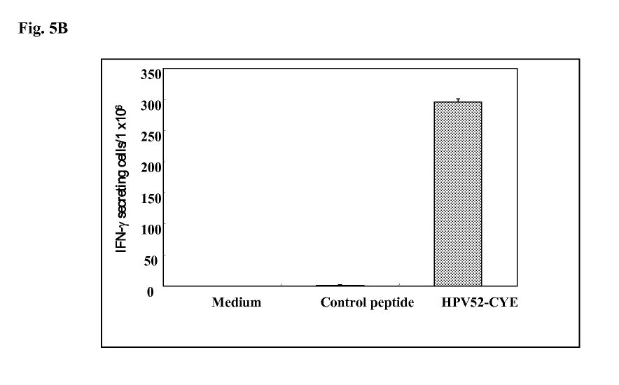

The ELISA results shown in FIG. 3 indicate that the MHC-peptide complex formation assay accurately determines whether a CTL epitope was restricted to a particular HLA class I allele. More specifically, stable HLA A2-β2-peptide complexes were detected in the presence of the two A2-restricted peptides but not the control peptide. See FIG. 3, left panel. Similarly, only the two A11-restricted peptides formed stable complex with HLA A11 heavy chain and β2 microglobulin. See FIG. 3, right panel.

Applying the above-described assay, a number of HLA A11-restricted epitopes were identified (relative binding %>30%). See FIG. 4. These epitopes were derived from either E6 or E7 of HPV16, HPV18, HPV58, and HPV58. Among them, the following three epitopes were novel: VVYRDSIPH (SEQ ID NO:1; 54-62 of HPV 18 E6); IMCLRFLSK (SEQ ID NO:2; 64-72 of HPV 52 E6; and KCLNEILIR (SEQ ID NO:3; 94-102 of HPV 58 E6).

### EXAMPLE 2

### Identification of HLA A24 Restricted CTL Epitopes Derived from HPV E7 Proteins

The HLA A24 heavy chain and β2-microglobulin were expressed and purified following the method described above. These proteins were incubated with synthetic peptides containing potential CTL-epitopes identified by the computational programs mentioned in Example 1. The formation of HLA A24-β2-peptide complexes was determined by the ELISA assay also described in Example 1 above.

The following two peptides were found to be novel HLA A24 restricted epitopes: VDLLCHEQL (SEQ ID NO:4; 23-31 of HPV 18 E7) and CYEQLGDSS (SEQ ID NO:5; 24-32 of HPV 52 E7).

### EXAMPLE 3

### Immunization of HLA-A11/A24 Transgenic Mice with HLA-A11/A24 Restricted Epitopes

C57BL/6-HLA-A11 transgenic mouse, derived from the Animal Technology Institute in Taiwan, was used in this study. 50 μg synthetic peptides having the amino acid sequence of VVYRDSIPH (A11VVY; SEQ ID NO:1) were formulated with incomplete adjuvant ISA-51 in the presence of a peptide MQWNSTTFHQTLQ (SEQ ID NO:17), which contains a T helper cell epitope derived from hepatitis B virus. The composition thus formed was injected subcutaneously into the HLA-A11 transgenic mouse two times every two weeks. Seven days after the final injection, splenotyes from the immunized mouse were isolated and cultured in a medium containing A11VVY. The level of IFN-γ secreted to the medium was determined by Enzyme-linked immunospot (ELISPOT) as described below.

A 96-well plate, having nitrocellulose membrane inserted in each well, was coated with 50 ml anti-IFN-γ antibody (clone MAB1-D1K, 10 μg/ml in 1×PBS, from Mabtech, Macka, Sweden) and was incubated at 4° C. for 24 hours. The plate was washed four times with PBS, and then incubated with 100 ml per well RPMI medium containing 10% fetal bovine serum (FBS) for 1-3 hours. Next, splenocytes were suspended in RPMI containing 10% FBS and 1×10⁵ or 5×10⁵ splennocytes (200 μl) were added to each well with or without A11VVY. The ELISPOT assay was performed in triplicate wells for each experimental condition. The plate was incubated in an incubator supplied with 5% CO₂ at 37° C. for 2 hours. After being washed for 2-3 times, the plate was incubated with avidin-peroxidase complex reagent for one hour at room temperature and then washed three times with 0.05% (w/v) Tween 20 in PBS and three times with PBS. 100 μl aminoethylcarbazole staining solution were then added to each well to develop stained spots. After 4-6 minutes, the plate was washed with running tap water to stop the reaction of developing the spots, which were counted by a computational image analysis.

Results obtained from the above assay show that a large amount of splenocytes derived from the HLA-A11 transgenic mouse immunized with A11VVY were IFN-γ secreting cells, indicating that these cells were activated by the peptide. See FIG. 5. To the contrary, little splenocytes derived from the same transgenic mouse immunized with a control peptide (WLSLLVPFV, SEQ ID NO:16) secret IFN-γ. These data clearly indicate that A11VVY is an HLA-A11 restricted CTL epitope. See FIG. 5A. Similarly, A24 CYE (CYEQLGDSS, SEQ ID NO:5) peptide incuded A24CYE-specific T cells to secrete IFN-γ in HLA-A24 transgenic mice. See FIG. 5B.

### EXAMPLE 4

### Induction of Cytotoxic T Lymphocyte (CTL) Effects with Peptide VVY in HLA-A11 Transgenic Mice

C57BL/6-HLA-A11 transgenic mice were immunized s.c. twice with peptide VVY (SEQ ID NO:1) or control peptide SSC (SEQ ID NO:12) together with a T helper cell epitope MQWNSTTFHQTLQ (SEQ ID NO:17), and incomplete adjuvant ISA-51. Seven days after the second immunization, splenocytes were harvested from the immunized mice, cultivated in vitro in the presence of peptide VVY or SSC (10 μg/ml) and rIL-2 (10 U/ml) for five days. The cells were then double stained with HLA-A11-peptide tetramer VVY-tet-PE or SCC-tet-PE and a FITC-conjugated anti-CD8 antibody to detect CD8⁺ T cells that bind to either VVY or SSC.

The tetramers were prepared as follows. Purified HLA-A11 heavy chains and β2-microglobulins were refolded in the presence of VVY or SSC in a refolding buffer containing 100 mM Tris-HCl (pH 8), 400 mM L-arginine, 2 mM EDTA, 5 mM reduced glutathione, 0.5 mM oxidized glutathione for 48 hrs at 4° C. The refolded HLA-A11/peptide complexes were biotinylated by BirA ligase (EC 6.3.4.15, Avidity, Colo., USA) in the presence of 0.4 mM of biotin and purified by gel filtration chromatography (S-200 column). The purified biotinylated monomeric HLA-A11/peptide complexes were then mixed with strptavidin-PE to form tetramers.

The tetramers thus prepared were incubated with the peptide-challenged T cells in a FACS staining buffer for 20 min at room temperature and a FITC conjugated anti-CD8 antibody was then added to the incubation system and kept on ice for 20 min. The stained cells were analyzed by flow cytometry (FACScalibur BD Bioscience, Calif., USA) in the presence of 1 μg/ml of propidium iodide (Sigma, Mo., USA) to exclude dead cells.

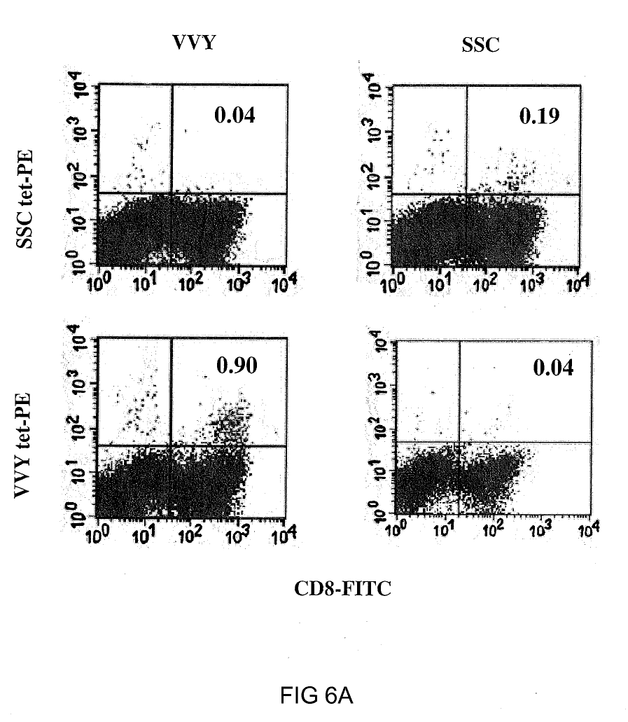

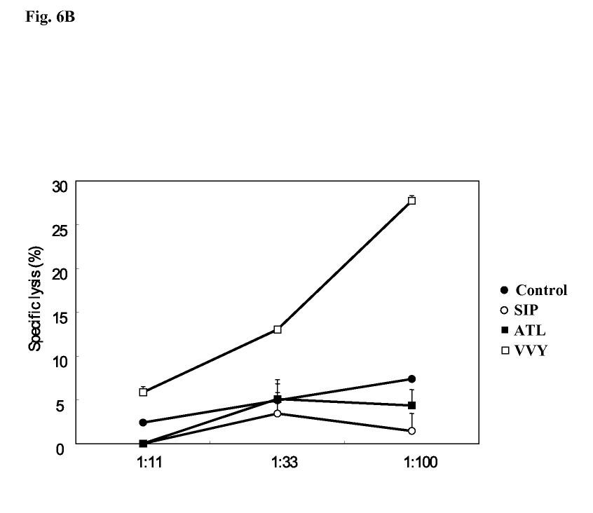

As shown in FIG. 6A, VVY induced VVY-specific CD8⁺ T cells in the transgenic mice while A11-SCC did not show this effect.

Next, the CTL effect induced by peptide VVY was examined as follows. HLA-A11 transgenic mice were immunized twice with peptide VVY, SIP, or ATL, all of which were derived from HPV 18, following the procedures described above in this Example. Splenocytes (2×10⁶/ml) isolated from the immunized mice were cultured with 10 μg/ml of either VVY or A11-SCC and 10 U/ml rIL-2 for at 37+ C. for 5 days. A standard chromium release assay was performed to determine cytolytic activity. In brief, T2/HLA-A11 cells were labeled with 100 μCi of ⁵¹Cr (PerkinElmer, Waltham, Mass., USA) for 1.5 hrs at 37° C. and washed afterwards three times with cold RPMI. The cultured splenocytes, used as effector cells, were mixed with 5×10³ the ⁵¹Cr-labeled T2/HLA-A11 cells at various ratios in triplicate wells in a U-bottom 96-well plate (Nunc, Rochester, N.Y., USA). After 4-5 hrs incubation, supernatants from each well were collected and the radioactivity contained therein was measured using a γ-counter (PARKARD, Ramsey, Mich., USA). The specific lysis rate was determined following the formula: Specific lysis rate (%)=(Sample_{cpm}−Spontaneous release_{cpm})/(Maximum release_{cpm}−Spontaneous release_{cpm})%.

As shown in FIG. 6B, VVY, but not SIP and ATL, induced CTL effects in the transgenic mice, indicating that this peptide is a CTL epitope.

### EXAMPLE 5

### Immunization of HLA-A11 Transgenic Mice with Antigen-Expressing DNA Plasmids

Plasmid pE3/19K/HPV18E6E7 was constructed following the conventional recombinant technology. This plasmid contains the E3/19K leader sequence and a nucleotide sequence encoding a fusion protein composed of the H-2K^{b}-restricted CTL epitope SII (derived from ovalbumin) and HPV18E6E7 polypeptide, which includes epitopes VVY, SIP, and ATL.

Female HLA-A11/K^{b} transgenic mice (6-8 week old) were immunized intramuscularly twice (at weeks 0 and 3) with 100 μg of plasmids pE3/19K/HPV18E6E7 and control plasmid pCIneo (does not express any antigen), both dissolved in 200 μL of PBS (pH 7.4). Seven to ten days after the second immunization, the mice were sacrificed and their splenocytes collected.

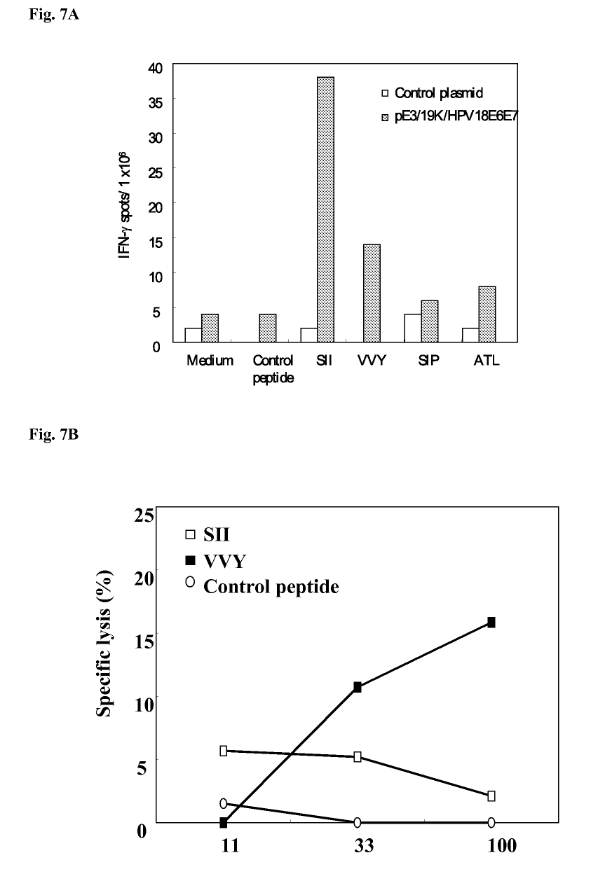

The splenocytes werestimulated with peptide VVY, SII, SIP, ATL, or a control peptide and the levels of IFN-γ secreted thereby were determined with the Enzyme-linked immunospot (ELISPOT) assay described in Example 1 above. The results thus obtained are shown in FIG. 7A. Peptides VVY and SII, but not SIP, ATL, and the control peptide, induced IFN-γ secretion, indicating that only VVY and SII activated the splenocytes (as evidenced by induction of IFN-γ secretion).

Next, peptide-specific CTL effects were examined by the chromium-release assay described in Example 4 above. As shown in FIG. 7B, only peptide VVY, but not SII and the control peptide, induced CTL effects. This result indicates that the a DNA plasmid expressing the VVY peptide can be used as a DNA vaccine to induce CTL responses in vivo.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. An isolated immunopeptide, comprising an amino acid sequence selected from the group consisting of:

| | |
|---|---|
| VVYRDSIPH, | (SEQ ID NO:1) |
| IMCLRFLSK, | (SEQ ID NO:2) |
| KCLNEILIR, | (SEQ ID NO:3) |
| VDLLCHEQL, | (SEQ ID NO:4) |
| and | |
| CYEQLGDSS, | (SEQ ID NO:5) |

wherein the immunopeptide includes 8-50 amino acid residues.

2. The isolated immunopeptide of claim 1, further comprising an amino acid sequence of QYIKANSKFIGITE (SEQ ID NO:6) or AKFVAAWTLK (SEQ ID NO:7).

3. The isolated immunopeptide of claim 1, further comprising an endoplasmic reticulum target sequence.

4. The isolated immunopeptide of claim 3, wherein the endoplasmic reticulum target sequence is selected from the group consisting of

| | |
|---|---|
| MRYMILGLLALAAVCSA, | (SEQ ID NO:8) |
| RYMILGLLALAAVCSA, | (SEQ ID NO:9) |
| MRAAGIGILTVAAAAAG, | (SEQ ID NO:10) |
| and | |
| MAGILGFVFTLAAAAAG. | (SEQ ID NO:11) |

5. The isolated immunopeptide of claim 2, further comprising an endoplasmic reticulum target sequence.

6. The isolated immunopeptide of claim 5, wherein the endoplasmic reticulum target sequence is selected from the group consisting of

| | |
|---|---|
| MRYMILGLLALAAVCSA, | (SEQ ID NO:8) |
| RYMILGLLALAAVCSA, | (SEQ ID NO:9) |
| MRAAGIGILTVAAAAAG, | (SEQ ID NO:10) |
| and | |
| MAGILGFVFTLAAAAAG. | (SEQ ID NO:11) |

7. An immunogenic composition comprising a carrier and an immunopeptide that includes an amino acid sequence selected from the group consisting of:

| | |
|---|---|
| VVYRDSIPH, | (SEQ ID NO:1) |
| IMCLRFLSK, | (SEQ ID NO:2) |
| KCLNEILIR, | (SEQ ID NO:3) |
| VDLLCHEQL, | (SEQ ID NO:4) |
| and | |
| CYEQLGDSS, | (SEQ ID NO:5) |

wherein the immunopeptide has 8-50 amino acid residues.

8. The immunogenic composition of claim 7, wherein the immunopeptide further includes an amino acid sequence of QYIKANSKFIGITE (SEQ ID NO:6) or AKFVAAWTLK (SEQ ID NO:7).

9. The immunogenic composition of claim 7, wherein the immunopeptide further includes an endoplasmic reticulum target sequence.

10. The immunogenic composition of claim 9, wherein the endoplasmic reticulum target sequence is selected from the group consisting of

| | |
|---|---|
| MRYMILGLLALAAVCSA, | (SEQ ID NO:8) |
| RYMILGLLALAAVCSA, | (SEQ ID NO:9) |
| MRAAGIGILTVAAAAAG, | (SEQ ID NO:10) |
| and | |
| MAGILGFVFTLAAAAAG. | (SEQ ID NO:11) |

11. The immunogenic composition of claim 8, wherein the immunopeptide further includes an endoplasmic reticulum target sequence.

12 The immunogenic composition of claim 11, wherein the endoplasmic reticulum target sequence is selected from the group consisting of

| | |
|---|---|
| MRYMILGLLALAAVCSA, | (SEQ ID NO:8) |
| RYMILGLLALAAVCSA, | (SEQ ID NO:9) |
| MRAAGIGILTVAAAAAG, | (SEQ ID NO:10) |
| and | |
| MAGILGFVFTLAAAAAG. | (SEQ ID NO:11) |

13. The immunogenic composition of claim 7, wherein the carrier is an adjuvant.

14. A method for enhancing an immune response against HPV, comprising administering to a subject in need thereof of an effective amount of a composition containing a carrier and an immunopeptide that includes an amino acid sequence selected from the group consisting of:

| | |
|---|---|
| VVYRDSIPH, | (SEQ ID NO:1) |
| IMCLRFLSK, | (SEQ ID NO:2) |
| KCLNEILIR, | (SEQ ID NO:3) |
| VDLLCHEQL, | (SEQ ID NO:4) |
| and | |
| CYEQLGDSS, | (SEQ ID NO:5) |

wherein the immunopeptide has 8-50 amino acid residues.

15. The method of claim 14, wherein the immunopeptide further includes an amino acid sequence of QYIKANSKFIGITE (SEQ ID NO:6) or AKFVAAWTLK (SEQ ID NO:7).

16. The method of claim 14, wherein the immunopeptide further includes an endoplasmic reticulum target sequence.

17. The method of claim 15, wherein the immunopeptide further includes an endoplasmic reticulum target sequence.

18. The method of claim 14, wherein the carrier is an adjuvant.

19. A method for treating HPV-associated disease, comprising administering to a subject in need thereof of an effective amount of a composition containing a carrier and an immunopeptide that includes an amino acid sequence selected from the group consisting of:

| | |
|---|---|
| VVYRDSIPH, | (SEQ ID NO:1) |
| IMCLRFLSK, | (SEQ ID NO:2) |
| KCLNEILIR, | (SEQ ID NO:3) |
| VDLLCHEQL, | (SEQ ID NO:4) |
| and | |
| CYEQLGDSS, | (SEQ ID NO:5) |

wherein the immunopeptide has 8-50 amino acid residues.

20. The method of claim 19, wherein the immunopeptide further includes an amino acid sequence of QYIKANSKFIGITE (SEQ ID NO:6) or AKFVAAWTLK (SEQ ID NO:7).

21. The method of claim 19, wherein the immunopeptide further includes an endoplasmic reticulum target sequence.

22. The method of claim 20, wherein the immunopeptide further includes an endoplasmic reticulum target sequence.

23. The method of claim 19, wherein the HPV-associated disease is cervical cancer or HPV infection.

24. The method of claim 19, wherein the carrier is an adjuvant.

25. An antibody specifically binding to a peptide having an amino acid sequence selected from the group consisting of:

| | |
|---|---|
| VVYRDSIPH, | (SEQ ID NO:1) |
| IMCLRFLSK, | (SEQ ID NO:2) |
| KCLNEILIR, | (SEQ ID NO:3) |
| VDLLCHEQL, | (SEQ ID NO:4) |
| and | |
| CYEQLGDSS. | (SEQ ID NO:5) |

26-29. (canceled)
